(19) United States
(12) Patent Application Publication
(10) Pub. No.: US 2003/0013142 A1
(43) Pub. Date: Jan. 16, 2003

(54) In vivo protein-protein interaction mapping

(75) Inventors: Pamela A. Silver, Cambridge, MA (US); Marc Damelin, New York, NY (US)

Correspondence Address: NIXON PEABODY LLP, 101 FEDERAL ST, BOSTON, MA, 02110, US

(73) Assignee: Dana-Farber Cancer Institute, Inc.

(21) Appl. No.: 10/188,393

(22) Filed: Jul. 2, 2002

Related U.S. Application Data
(63) Continuation of application No. PCT/US01/02717, filed on Jan. 26, 2001.
(60) Provisional application No. 60/178,183, filed on Jan. 26, 2000.

(51) Int. Cl. G01N 33/567; G01N 33/53; G01N 33/569

(52) U.S. Cl. 435/7.21; 435/7.31

(57) ABSTRACT

The present invention is directed to the use of fluorescence resonance envery transfer (FRET) to screen for protein-protein interactions. More specifically, this invention provides a method to screen for and identify proteins that interact with a protein of interest. The method involves using DNA encoding the protein of interest tagged with one member of a fluorescent protein pair, and DNA sequences encoding a library of proteins to be screened, in which the proteins are tagged to the other member of the fluorescent pair. The protein of interest is co-expressed in cells with the library of proteins to be screened, such that single cell expresses the protein of interest and a single representative of the tagged library. This population of cells is screened by flourescence resonance energy transfer. Cells where interations have occurred are selected, allowing identification of the protein that interacts with the protein of interest.

This invention was supported by National Institutes of Health Grant GM36373 and the government of the United States has certain rights thereto.

### FIELD OF THE INVENTION

The present invention is directed to the use of fluorescence resonance energy transfer (FRET) to screen for protein-protein interactions.

### BACKGROUND OF THE INVENTION

Advances in genomics have resulted in the discovery and identification of numerous genes encoding proteins. These advances have made it possible to obtain transcripts for a range of proteins. However, one of the problems that has developed is that while many proteins are being classified based on the sequence relation of a protein to a class of proteins, the exact function of such proteins is often not known. Even when looking at proteins having known functions, their ability to bind to and interact with other proteins is frequently not known.

One particular area of interest is the ability of one protein to bind to another. However, identifying relevant interacting pairs has proven difficult. For example, even if it can be shown that protein A will bind to protein B, typically other proteins will also bind to protein A. In return, protein B may bind to many other proteins in addition to protein A. In addition, the fact that two proteins are able to bind to each other in vitro does not indicate that they will do so in a living system where there is competition from other proteins. Consequently, it would be desirable to have a method to be able to determine when and where in a cell specific proteins do interact with each other.

There have been many attempts to try to deal with such problems. These attempts have included using cells in which a particular protein is over expressed or cell systems where the protein is generally not expressed at all. In such systems, the protein interactions that can be viewed using available techniques are typically limited to stable interactions. It would be desirable to have a method where one can use the methodology in a wide range of cells and to identify transient as well as stable interactions.

Fluorescence resonance energy transfer (FRET) has been used to visualize a variety of interactions, e.g., interactions between nucleic acids, interactions involving sugars, and in some instances interactions between proteins (Stryer, 1978). In FRET, an excited donor fluorophore can transfer energy to an acceptor under very specific conditions, which include a donor-acceptor separation of ˜10-100 Å and a specific relative orientation of donor and acceptor (Clegg, 1996). FRET can be detected by exciting the donor and measuring an increase in acceptor emmission or a decrease in donor emission. Most FRET protein studies have been conducted in vitro because of the inherent difficulty of labeling two proteins in vivo, but recently mutant derivatives of the Green Fluorescent Protein (GFP) with spectral overlaps suitable for FRET have been developed (Heim and Tsien, 1996). The preferred donor-acceptor pairs include EBFP-EGFP (enhanced blue and green fluorescent proteins) and ECFP-EYFP (enhanced cyan and yellow fluorescent proteins). In those situations involving proteins, FRET has previously been used to look at specific proteins to confirm known interactions, not for screening. (Miyawaki et al., 1997; Day, 1998; Mahajan et al., 1998). One of the limitations of FRET has been that while positive results can demonstrate that an interaction between two proteins has occurred, a negative result does not necessarily provide information about lack of an interaction. The reason for this is the relatively restricted distance over which FRET can occur. For example, there is a distance of about 40 Å for interactions between EBFP and EGFP and about 50Å for ECFP and EYFP (Heim and Tsien 1996) Thus, if the two paired fluorescent proteins are not situated sufficiently close to each other, even though two proteins are interacting, a negative result will be seen. Thus, this technique has been limited to confirmatory interactions. However, it would be extremely desirable to have a system to readily screen a wide number of potential proteins to determine whether or not they interact.

### SUMMARY OF THE INVENTION

We have now discovered a method to screen for and identify proteins that interact with a protein of interest. The method involves using DNA encoding the protein of interest tagged with one member of a fluorescent protein pair, and DNA sequences encoding a plurality of proteins to be screened, wherein the proteins are tagged to the other member of the fluorescent pair.

The DNA encoding the protein of interest is used to transform a plurality of cells, i.e., expression system. Thereafter, DNA encoding the protein being screened is expressed in those transformed cells. The cells are screened by fluorescence resonance energy transfer (FRET) to determine whether an interaction has occurred. Cells where interactions have occurred are selected and in this manner a protein that interacts with the protein of interest is identified.

### BRIEF DESCRIPTION OF THE DRAWINGS

FIGS. 1A-C shows energy transfer between ECFP and EYFP in living yeast cells. FIG. 1A shows lysates from cells expressing either NLS-ECFP and NLS-EYFP, or the chimera NLS-ECFP-EYFP. The lysates were resolved by SDS-PAGE, transferred to nitrocellulose membrane, and probed with anti-GFP antibody. FIG. 1B shows cells expressing either NLS-ECFP or NLS-EYFP grown to log phase and analyzed by fluorescence microscopy with filter sets for ECFP (440 nm excitation, 480 nm emission) or EYFP (500 nm excitation, 545 mn emission), with Nomarski optics. FIG. 1C shows cells expressing either NLS-ECFP and NLS-EYFP, or the chimera NLS-ECFP-EYFP, viewed with the FRET filter set (440 nm excitation, 535 nm emission).

FIGS. 2A-E demonstrates that importin Pse1 and the nucleoporin Nic96 interact by FRET. (FIG. 2A) Lysates from cells expressing either Nic96-EYFP, Pse1-ECFP, or Pse1-ECF+Nic96-EYFP, were resolved by SDS-PAGE, transferred to nitrocellulose membrane, and probed with anti-GFP antibody. (FIG. 2B) Cells expressing Pse1-ECFP or Nic96-EYFP fusions were viewed with the CFP or YFP filters, respectively, and with Nomarski optics. (FIG. 2C) Cells expressing Nic96-EYFP, Pse1-ECFP, Pse1-ECFP+Nic96-EYFP, or Pse1-ECFP+Nup59-EYFP were viewed with the FRET filter set. (FIG. 2D) Intensity profiles of cells in (FIG. 2C) were obtained by plotting the FRET intensity along a line drawn through a given cell. Profiles are shown for a cell expressing Pse1-ECFP (solid line), Pse1-ECFP+Nic96-EYFP (dashed line), and Pse1-ECFP+Nup59-EYFP (dotted line). The white line adjacent to each cell in (FIG. 2C) indicates the line along which that profile was calculated. (FIG. 2E) Digital images of the strains in (FIG. 2C) were quantitated to yield FRET Values (see Examples). The FRET Value represents the average of the FRET/ECFP ratio at the nuclear envelope in 15-20 cells per strain, normalized such that the FRET Value for Pse1-ECFP equals zero. The averages of the FRET and ECFP intensities for the same cells are also shown (Avg. FRET and Avg. ECFP, respectively). All values are shown±standard deviation.

FIG. 3 shows that Pse1-nucleoporin FRET signals represent sensitized emission. Cells expressing Pse1-ECFP, Pse1-ECFP+Nic96-EYFP, or Pse1-ECFP+Nup188-EYFP were viewed with the FRET filter set either before (−) or after (+) a 15-second exposure to the YFP filter set.

FIGS. 4A-B shows that the Pse1-Nup1 FRET interaction is not observed in nup1-8. In FIG. 4A cells expressing Nup1-EYFP or Nup1-8-EYFP were grown to log phase and viewed with the YFP filter set and Nomarski optics. FIG. 4B shows cells expressing Pse1-ECFP, Pse1-ECFP+Nup1-EYFP, or Pse1-ECFP+Nup1-8-EYFP viewed with the FRET filter set.

FIGS. 5A-B analyzes the Pse1-Nic96 interaction. (FIG. 5A) Wildtype, PSE1-ECFP, or rna1-1 PSE1-ECFP cells were grown to log phase at 25° C., and half of each culture was shifted to 37° C. for 75 minutes. Whole-cell lysates were incubated with anti-GFP beads, based on Seedorf et al., 1999. The precipitated complexes were resolved by SDS-PAGE, transferred to nitrocellulose membrane, and probed with anti-GFP (top) or anti-Nic96 (bottom). (FIG. 5B) Wildtype cells expressing Pse1-GFP (PSE1-GFP) or mutant nic96-1 cells expressing Pse1-GFP (nic96-1 PSE1-GFP) were grown at 25° C. and viewed with standard GFP filters and Nomarski optics.

### DETAILED DESCRIPTION OF THE INVENTION

The method of the present invention can be used to screen for and identify proteins that interact with a protein of interest.

FRET has been used to confirm protein-protein interactions by the engineering of GFP derivatives that can be fused to proteins of interest. For example, using two fluorescent proteins linked by calcium-binding domains to monitor calcium concentrations in HeLa cells (Miyawaki et al., 1997). Subsequent studies, also in mammalian cell lines, have characterized interactions of the Pit-1 transcription factor (Day, 1998) and of Bcl-2 family proteins (Mahajan et al., 1998). In these cases, the fusions were overexpressed compared to physiological levels and could not be tested for overall functionality.

The present method screens for proteins that interact with a protein of interest. Specifically, the protein of interest is tagged with one member of a fluorescent protein pair. The other member of the fluorescent protein pair is tagged to the protein(s) being screened. One preferred way of tagging the protein is by using a cassette that can be used to insert the nucleic acid encoding the protein so that when expressed it is tagged with the desired fluorescent protein.

The proteins can be expressed in any desired cell (expression system). Preferred cells include mammalian cells such as human cells, monkey cells, murine cells (e.g., rat, mice), rabbit cells, yeast cells, insect cells, etc.

Numerous proteins have a variety of different binding partners. Proteins can have multiple protein binding domains with each domain responsible for different functions. Some proteins form stable associations with one or several other proteins, such as the beta and gamma subunits of heterotrimeric G proteins. Additionally, some proteins may associate with another protein(s) under certain conditions. For example, the alpha subunit of heterotrimeric G proteins associates with the beta/gamma complex only when the alpha subunit itself is bound to GTP. Further, some proteins may transiently associate with another protein, such as the transient interaction between a kinase and its target substrate. Furthermore, some proteins function as part of a larger protein complex. For example, RNA polymerase, the enzyme that actually synthesizes the new strand of RNA during transcription, is only one of many proteins found in the transcription complex. While many members of the transcription complex form a stable core and are always present (such as the polymerase itself), other members associate with the complex only under certain conditions (e.g. before the polymerization begins). Some protein interactions may be specific to certain physiological conditions, such as nutrient conditions, stages of development, or cancerous transformation.

Moreover, there are instances where the same protein binding site is promiscuous, i.e., will bind to multiple proteins. For example, modular protein binding domains are regions of about 60 to 200 amino acids, such as src homology 2 (SH2), src homology 3 (SH3), phosphotyrosine binding (PTB), WW, PDZ, 14.3.3, WD40, EH, Lim, etc. Ab1 is a kinase with a corresponding binding site that forms a complex with a number of adaptor proteins. However, it is believed that one protein pair predominates in the normal wild type situation. Moreover, different protein pairs may predominate at different times (e.g., in developing cells, healthy vs. malignant cells, etc.). These examples represent only some of the many situations in which proteins interact.

The method of the present invention can be used to screen for and identify proteins that interact with a protein of interest. The method comprises a number of components. First, a cassette to tag the protein of interest with one of a pair of fluorescent proteins (e.g. ECFP). Second, a cassette in which to construct a library of putative target proteins tagged with the complementary fluorescent protein (e.g. EYFP). Third, an expression system in which to conduct the screen, comprising cells for expression of the tagged proteins and vectors compatible with said cells for carrying the tagged-protein cassettes.

The cassettes carrying the genes encoding the tagged proteins are constructed and introduced into an appropriate vector. Then the tagged protein of interest (i.e. the vector carrying a cassette) is expressed in every cell of the target cell population. Finally, the vector library containing the tagged putative target protein cassettes is co-expressed in the target cell population. The transformed cells are screened to identify any protein-protein interactions by FRET.

The pair of fluorescent tags that are used in the present invention are the complementary pair, namely, when the proteins they encode are located physically within an appropriate distance of one another, FRET occurs. Typically, one of the tags in a pair is fused to the protein of interest and the other tag of the pair is fused to the putative target protein. Pairs of fluorescent tags include any two genes encoding fluorescent proteins with suitable spectral overlaps to undergo FRET. For example, GFP/BFP and ECFP/EYFP. One can use any fluorescent protein pair. However, ECFP and EYFP are presently preferred over EBFP and EGFP because of several advantages for intermolecular FRET measurements. ECFP is brighter than EBFP and less sensitive to photobleaching. ECFP-EYFP also has a larger critical radius than EBFP-EGFP (˜50 Å versus ˜40 Å), indicating that ECFP-EYFP FRET has a longer detection range.

In general, the detection of ECFP-EYFP FRET requires that the fluorophores are within 50-60 Å of each other, based on the ECFP-EYFP critical radius of ˜50 Å. Since each fluorophore is buried inside the ECFP/EYFP barrel structure (Ormo et al., 1996; Yang et al., 1996), this maximum inter-fluorophore distance translates to a maximum separation between ECFP and EYFP of only 25-35 Å. Therefore, a FRET signal between transport receptor-ECFP and nucleoporin-EYFP fusions indicates a high probability that 2 proteins are binding directly.

The tags are preferably constructed to contain a multi-site cloning linker region at either end, to assist introduction of the tag into the cassette for fusion to the gene encoding the particular protein.

The cassettes for expressing the protein of interest and the putative target proteins have several features: a gene encoding a fluorescent tag; a gene encoding a polypeptide of interest; and a multi-site cloning linker region. Additionally, the cassettes can include a second tag as well as a promoter.

The cassettes for expressing the two classes of proteins (the protein of interest and the population of target proteins) may fuse the fluorescent tag to the given protein in one of several places.

The cassettes may introduce the fluorescent tag onto the N-terminus or the C-terminus of the given protein.

Alternatively, the fluorescent tag can be fused to the protein of interest and the putative target proteins to enhance the performance of any interaction. One important factor which influences the FRET reaction between two fluorescent molecules is the distance between the fluorophores. The distance restriction is specific for each pair of fluorescent proteins (Heim and Tsien, 1996). As discussed above, for ECFP/EYFP (exemplified below) a maximum distance of 25-35 angstroms is required to observe FRET. To enhance the performance of the screen, given this distance requirement, several modifications can be used. First, one can generate two cassettes bearing the protein of interest, one of which fuses the fluorescent tag to the protein's N-terminus and the other of which fuses the fluorescent tag to the C-terminus. Both tagged versions of the protein of interest can then be used to screen a library for interacting proteins. Similarly, two cassettes for the library of putative target proteins can be generated, one set of which creates N-terminal fusion proteins and the other of which creates C-terminal fusion proteins. Again, both libraries can be screened with a tagged protein of interest.

The placement of the fluorescent tag can be further refined using additional information regarding the protein of interest. For example, structural information may be available which allows placement of the tag to be manipulated to increase the likelihood of interaction with other proteins. For example, it may be known that a certain protein binding site falls within the middle portion of the linear amino acid sequence for a protein but lies on the surface of the folded protein. In such an instance, it may be of interest to insert the fluorescent tag conformationally proximal to that site of the protein of interest, rather than at one of the protein's termini. This can be done with a linker amino acid sequence. This linker sequence is preferably neutrally charged and the amino acids selected are chosen so they do not create much steric hinderance which can effect the normal folding pattern of the protein. Tagging the interior portions of the putative target proteins may be preferable when screening a limited number of putative target proteins, particularly if the target falls within a class of proteins with some structural information available.

In another modification, the cassette may contain the entire protein of interest or only a part of the protein of interest, typically a domain of interest. Protein domains are modular units from which proteins are constructed, and usually comprise a section of polypeptide chain that contains between 50 and 350 amino acids. While small proteins may contain only a single domain, larger proteins can contain a number of domains, which are usually connected by relatively open lengths of polypeptide chain. In another embodiment the protein may be constructed to delete certain domains to screen and identify what proteins interact with what domains.

The cassettes are preferably constructed to contain a multi-site cloning linker region at either end, to assist in introduction of the cassette into the appropriate vector.

The cassettes for expressing the protein of interest and the population of target proteins can include a second tag (such as a marker, e.g., hemagglutinin) for monitoring the protein independent of its fluorescent tag.

The cassettes for expressing the protein of interest and the population of target proteins can include a promoter for expression of the protein. Such promoters include constitutive and inducible promoters as well as the native promoter of the genes encoding the individual tagged proteins. Alternatively, the promoter can be an integral part of the vector. The promoter chosen are selected based upon the host cell which the protein is expressed in. Suitable promoters include cytomegalovirus (CMV) intermediate early promoter, a viral LTR such as the Rous sarcoma virus LTR, HIV-LTR, HTLV-1 LTR, the simian virus 40 (SV40) early promoter, E. coli UA5 promoter, the herpes simplex tk virus promoter, and yeast promoters such as those naturally associated with GAL4, TPI1 (triose phophate isomerase), PGK1 (phophoglycerate kinase), PYK1 (pyruvate kinase), TKH1, TDH2, and TDH3 (glyceraldehyde phosphate dehydrigenase or triose phosphate dehydrogenase), and ENO1 (enolase 1).

The genes encoding the protein of interest and the library of putative tagged proteins can be chosen from any organism for which the necessary molecular biology techniques are available. The population of putative target proteins can include all open reading frames for an organism, or those expressed under certain conditions, or a subset of open reading frames of particular interest for the given protein.

The expression system in which the screen is conducted comprises cells for expression of the tagged proteins and vectors compatible with said cells for carrying the tagged-protein cassettes. Thus, for expression in a mammalian cell, a vector which will transform and express in mammalian cells is required. Any system which allows introduction and expression of DNA can be used.

Particularly suitable expression systems include for example, but are not limited to, the yeast Saccharomyces cerevisiae, mammalian cell lines, and bacteria including Escherichia coli and pathogenic bacteria.

The yeast system offers several advantages for in vivo FRET experiments. First, fluorescent proteins are produced from integrated genomic fusions, so that each is expressed under its own promoter and is the only copy of that protein in the cell. Thus, there is less likelihood of mislocalization due to abnormally high concentrations of the protein or competition for binding sites between tagged fusion proteins and untagged endogenous proteins. Second, the functionality of a fusion protein can be tested in a genetic background where it is required for cell viability. Functionality ensures that the behavior of the fusion protein reflects that of its native counterpart. Third, studies in mutants can reinforce the physiological relevance of individual protein-protein interactions.

The expression system can be homologous, for example a screen conducted in yeast for yeast proteins that interact with a yeast protein of interest, or the expression system can be heterologous, for example a screen for mammalian proteins such as human proteins that interact with a human protein of interest can be conducted in a yeast cell.

The particular vector chosen will depend upon the host cell used.

Vectors include chemical conjugates, plasmids, phage, etc. The vectors can be chromosomal, non-chromosomal or synthetic. Commercial expression vectors are well known in the art, for example pcDNA 3.1, pcDNA4 HisMax, pACH, pMT4, PND, etc. Preferred vectors include viral vectors, fusion proteins and chemical conjugates. Retroviral vectors include Moloney murine leukemia viruses and psuedotyped lenteviral vectors such as FIV or HIV cores with a heterologous envelope. Other vectors include pox vectors such as orthopox or avipox vectors, herpesvirus vectors such as a herpes simplex I virus (HSV) vector (Geller, A. I. et al., (1995), J. Neurochem, 64: 487; Lim, F., et al., (1995) in DNA Cloning: Mammalian Systems, D. Glover, Ed., Oxford Univ. Press, Oxford England; Geller, A. I. et al. (1993), Proc Natl. Acad. Sci.: U.S.A. 90:7603; Geller, A. I., et al., (1990) Proc Natl. Acad. Sci USA 87:1149), adenovirus vectors (LeGal LaSalle et al. (1993), Science, 259:988; Davidson, et al. (1993) Nat. Genet 3: 219; Yang, et al., (1995) J. Virol. 69: 2004) and adeno-associated virus vectors (Kaplitt, M. G., et al. (1994) Nat. Genet. 8: 148).

The introduction of the gene into the host cell can be by standard techniques, e.g. infection, transfection, transduction or transformation. Examples of modes of gene transfer include e.g., naked DNA, CaPO₄ precipitation, DEAE dextran, electroporation, protoplast fusion, lipofection, cell microinjection, and viral vectors, adjuvant-assisted DNA, gene gun, catheters, etc.

The vectors for expressing the protein of interest as well as the library of putative target proteins can contain any features compatible with the cell expression system, including for example selectable markers. Additionally, the vector can contain a promoter if none is present in the cassette. Again, a variety of promoters can be used, including constitutive and inducible promoters as well as the native promoter of the genes encoding the individual tagged proteins.

The cell population in which the FRET interaction screen is performed can be manipulated such that each cell expresses the protein of interest and one member of the target population. This can be arranged, for example, by first introducing the vector carrying the gene encoding a protein of interest into the total population, and then introducing the library such that any single cell carries only one member of the target population.

In an alternative embodiment the gene encoding the protein of interest can be overexpressed.

FRET can be detected by any method, including fluorescent activated cell sorting and microscopy.

The transfer of energy from one fluorescent molecule to another occurs when the emission spectrum of the donor overlaps with the excitation spectrum of the acceptor. Thus, one excites the donor-acceptor complex at the excitation wavelength of the donor and the energy emitted by the donor is transferred to the acceptor, whereupon the acceptor is itself excited and emits energy at its emission wavelength. The efficient transfer of energy generally requires that the donor and acceptor molecules are within approximately 10-100 angstroms of each other (depending upon the particular molecules) and have a specific relative orientation. To detect the transfer of fluorescent energy, one typically excites a sample at the excitation wavelength of the donor fluorophore and measures the emission of fluorescence at the emission wavelength of the acceptor fluorophore.

Any pair of fluorophores for which the emission spectrum of the donor overlaps the excitation spectrum of the acceptor can be used in the present invention. Typically, the donor fluorophore tag is fused to the protein of interest and the acceptor fluorophore tag is fused to the putative target protein. Pairs of fluorescent tags include any two genes encoding fluorescent proteins with suitable spectral overlaps to undergo FRET. For example, GFP/ BFP (critical radius of approximately 40 angstroms) and ECFP/ EYFP (critical radius of approximately 50 angstroms). A preferred pair of fluorescent tags is one having a higher critical radius. For example, ECFP/EYFP as the efficiency of energy transfer is maintained at a greater distance.

The tags are preferably constructed to contain a flexible linker region at either end, to assist introduction of the tag into the cassette for fusion to the gene encoding the particular protein.

FRET can be detected by any method that allows exictation of the sample at the donor fluorophore's wavelength and detection of emission at the acceptor fluorophore's wavelength. For example, samples can be assayed by fluorescence microscopy. Another detection method is the use of a fluorimeter, which directly measures emmission; this technique is currently best applied to cell-free interactions. A third alternative, particularly useful for screening live cells carrying a large pool of clones, is the use of fluorescence activated cell sorting (FACS). Using FACS analysis, one can separate positive cells from negative cells, allowing their convenient recovery. Another approach is the use of newly developed microscopes which combine the high quality optics of fluorescence microscopy with the detection capacities of fluorescent activated cell scanning devices, allowing one to isolate and recover single positive cells which remain viable.

Quantitation of microscope images allows analysis at a particular subcellular location. This method optimizes the signal-to-noise ratio by excluding the regions that contributes only background signal. The normalization of FRET intensity by ECFP intensity corrects for cell-to-cell variation as well as systematic errors.

Domains are usually constructed from a section of polypeptide chain that contains between 50 and 350 amino acids. They are typically modular units from which proteins are constructed. While small proteins may contain only a single domain, larger proteins contain a number of domains, which are usually connected by relatively open lengths of polypeptide chain.

A polypeptide chain can form many different structures. The conformation adopted by a polypeptide chain depends on the amino acid sequence.

For example, a 300 amino acid chain, when folded into a well-packed globular structure, has a diameter of 43 angstroms. When the same chain is folded into other conformations, however, its dimensions alter radically: a square of beta-pleated sheet has a width of 70 angstroms, a triple-helix structure is 290 angstroms long, and a single alpha helix is 450 angstroms. Thus, the size of a protein depends on its three-dimensional packing of a protein. While the three-dimensional structure cannot be precisely predicted from its amino acid sequence, predictions can be made based upon homology with known proteins. In this manner care can be taken in placing the fluorescent protein tag. For example, as discussed above, adjacent to the domain of interest. Alternatively, using multiple cassettes so that the proteins of interest will be expressed with the tag placed at multiple spots, e.g., carboxy, amino, and internal intervals. By using multiple tagged versions, one can reduce the number of false negatives that can occur because the two tags are too far apart for FRET to occur.

The method of the present invention can be used in a variety of applications.

One can use the method of the present invention to independently confirm a protein-protein interaction that was identified using other methods (e.g. co-immunoprecipitation or in vitro binding). Similarly, one can use this method with two proteins known to interact to further define the interacting regions, by placing the tags at different sites on each partner and/or by expressing portions of the protein(s). Such an approach can be especially useful for large multi-domain proteins.

One can also use the method of the present invention to screen for interactions for a protein of interest with a range of target proteins. For example, the population of putative target proteins can include all open reading frames for an organism, or those expressed under certain conditions, or a subset of open reading specifically of interest for the protein of interest, such as interactions between exportins and nucleoporins, between kinases and adaptors, assembly of tubulin into microtubules, G protein subunits, etc.

In one embodiment one can look at the temporal occurrences of interactions because FRET permits dynamic measurements. Thus, one can identify transient interactions as well as more permanent interactions. In certain instances, one can also identify changes in interactions by looking at fluorescent changes resulting from disassociation of proteins as well as from binding, e.g., quenching of the acceptor fluorescence and emission at the donor's wave length.

One can also use the method of the present invention to compare a protein's interactions under different conditions. For example, the interacting proteins in a given protein pair may change during the cellular transformation process which leads to cancer. Similarly, there are examples of proteins (e.g. the homeodomain proteins) which bind different partners at different stages of development, giving the proteins different functions which are needed at different stages.

Viral infection and bacterial pathogenesis are other examples of differing conditions under which to examine a protein's interactions. One can use the method of the present invention to address what host cell protein(s) a bacterial or viral pathogen protein interacts with during its invasion, and whether these interactions change over the course of the pathogenic process. For example, the HIV nef protein interacts with cellular proteins to reduce immune function. One can tag the nef protein and identify the target proteins that interact with this protein. One can similarly tag other viral pathogen proteins or bacterial pathogen proteins in an analogous manner.

In yet another embodiment, one can recover the target gene from a positive cell. Methods for identifying and recovering target genes during screening techniques are well known in the art and specific to the system in which the screen is conducted. For example, if a screen is conducted in the yeast Saccharomyces cerevisiae, a plasmid bearing the gene of interest is readily recovered from the positive cell by isolating total DNA, transforming it into either bacteria or yeast, and selecting the transformed cells for a property associated with the plasmid (such as a nutritional or antibiotic marker).

One can also use this method to determine protein-protein interactions within a multi-component protein complex. Many cellular processes are governed by large multimeric protein machines, including, for example, transport of nascent polypeptides into the endoplasmic reticulum, DNA replication, protein degradation by the proteosome, and trafficking of molecules into and out of the nucleus.

For example, the transport of macromolecules between the nucleus and cytoplasm is crucial for controlling cellular processes such as gene expression, cell division, and signal transduction (Nigg, 1997). An elaborate system has evolved to regulate the import and export of proteins and the export of RNAs (Corbett and Silver, 1997).

Proteins and RNAs are transported through nuclear pore complexes (NPC) embedded in the nuclear envelope. Vertebrate NPCs are ˜125 Mda measuring ˜200 nm in length and ˜125 nm in diameter, while yeast NPCs are approximately 60% as large (Fahrenkrog et al., 1998). Electron microscopy has revealed the NPC's eightfold rotational symmetry and features referred to as cytoplasmic filaments and nuclear baskets (reviewed in Stoffler et al., 1999). Each NPC is composed of multiple copies of different nucleoporins (nups), many of which have repeat regions of the peptides FG, FXFG, or GLFG (Fabre and Hurt, 1997). The functions of particular nucleoporins have been studied by biochemical and genetic analyses.

Transport through the NPC is mediated by a family of transport receptors (importins and exportins) that share sequence homology in the Ran-binding domain (Wozniak et al., 1998). In the prevailing model, interactions between transport receptors and the Ran GTPase control the directionality of transport (Izaurralde et al., 1997; Richards et al., 1997). Fourteen transport receptors in the budding yeast Saccharomyces cerevisiae and many homologs in higher eukaryotes have been identified (reviewed in Wozniak et al., 1998). Many of these receptors have been matched with a specific import or export function. Importins and exportins recognize their respective protein cargoes via nuclear localization signals (NLS) or nuclear export signals (NES). Substrates bearing the canonical lysine-rich NLS, originally identified in the Simian Virus 40 Large T-antigen (Kalderon et al., 1984), are recognized by the Srp1/Kap95 heterodimer (importin-α/importin-β in mammalian cells) (Gorlich et al., 1995; Radu et al., 1995). Substrates with other localization signals are recognized by different receptors; for example, the yeast transcription factor Pho4 is recognized by both the importin Pse1/Kap121 (Kaffman et al., 1998) and the exportin Msn5 (Kaffman et al., 1998), depending on the phosphorylation state of Pho4.

The mechanism of translocation through the NPC involves interactions between receptors and nucleoporins. Studies in mammalian, Xenopus and yeast systems have identified specific receptor-nucleoporin contacts by in vitro binding and co-immunoprecipitation experiments (reviewed in (Ohno et al., 1998)). However, as discussed above, the in vitro results may not reflect the specificity of receptor-nucleoporin interactions in the context of the NPC in vivo, especially in light of the FG/FXFG/GLFG repeat regions shared by many nucleoporins. In the co-immunoprecipitation experiments, the detected interactions are not necessarily direct. In addition, many dynamic receptor-nucleoporin contacts may be too unstable for detection by these assays.

Accordingly, one can use the present method, e.g., ECFP-EYFP FRET system to screen for and identify the interactions between transport receptors and nucleoporins in an expression system such as yeast cells, as more fully demonstrated in the examples set forth below.

### EXAMPLES

Plasmid Construction

To construct pRS316-NLS-ECFP (pPS1887) and pRS314-NLS-EYFP (pPS1888), the ADH1 promoter, a duplex oligonucleotide encoding the SV40 T-antigen NLS, PCR-amplified ECFP or EYFP, and the NUF2 3′ UTR (Kahana et al., 1995), were cloned into the shuttle vectors pRS314 and pRS316 (Sikorski and Hieter, 1989). ECFP was cloned into pRS316-NLS-EYFP to generate pRS316-NLS-ECFP-EYFP (pPS 1889). The XhoI/KpnI fragment of pPS1887, containing ECFP and the 3′ UTR, was sublconed into pRS304 to generate an integrating vector with the TRP1 marker for C-terminal ECFP fusions (pPS1890). The XhoI/KpnI fragment of pPS1888 was sublconed into pRS306 to generate an integrating vector with the URA3 marker for C-terminal EYFP fusions (pPS1891).

DNA encoding a C-terminal fragment of each nucleoporin was amplified by PCR and ligated into pPS1891, to generate the NUP-EYFP integration vectors (pPS1892-1907). Each plasmid was linearized at a site within the NUP fragment to target integration at that locus. The PSE1 fragment was subcloned from pPS1538 to create PSE1-ECFP (pPS1910), and the MSN5 fragment was subcloned from pPS1721 to create MSN5-ECFP (pPS1912).

Strain Construction

Yeast strains were transformed with linearized vectors by the lithium acetate method. The wild-type FY23 (Winston et al., 1995) is a haploid S288C strain. Individual transformants were checked for expression of ECFP and EYFP fusions by microscopy and by Western blotting with anti-GFP antibody. The NUP-EYFP strains (PSY1831-1843, in the same order as listed in Table 1) were transformed with pPS1910 and pPS1912 to generate panels of strains with two fusions: PSE1-ECFP/NUP-EYFP (PSY 1844-PSY1856) and MSN5-ECFP/NUP-EYFP (PSY1870-1882). FY23 was transformed with pPS1910 and pPS1912 to create PSE1-ECFP (PSY1828) and MSN5-ECFP (PSY1830).

Microscopy

Cells were observed with a Nikon Diaphot-300 epifluorescence microscope with a 100-Watt mercury lamp, a 60×1.4 NA Plan-APO objective, Nomarski optics, and the following filter sets (Omega Optical, Brattleboro, Vt.): CFP 440 nm/20 nm excitation filter, 455 nm longpass dichroic filter, 480 nm/30 nm emission filter; YFP 500/25 nm excitation, 525 nm longpass, 545/35 nm emission; FRET 440/20 nm excitation, 455 nm longpass, 535/25 nm emission. Images were captured with a liquid-cooled CCD camera (Photometrics, Tuscon, Ariz.) equipped with a KAF-1400 chip, operated by the MetaMorph Imaging System (Universal Imaging Corp., West Chester, Pa.) and a Model D122 shutter driver (UniBlitz, Rochester, N.Y.).

Cells were grown at 25° C. to mid-log phase in synthetic complete or dropout medium, transferred to slides and examined immediately. Camera exposures were 1-2 seconds with the FRET and YFP filter sets and 4-5 seconds with the CFP filter set. Settings were identical for all images in a given experiment.

FRET signal was quantitated with a two filter-set system in order to normalize the FRET intensity for ECFP (donor) concentration in each cell. Two-filter set quantitation methods have been discussed extensively by Gordon et al., 1998. Digitized images were captured first with the FRET filter set (2 sec) and then with the CFP filter set (5 sec). Images were analyzed with the MetaMorph system by highlighting the nuclear envelope in the CFP image with the Threshold function, and calculating the average pixel intensity per area. The average intensity for the same region of the corresponding FRET image was then calculated. The ratio of average intensities was obtained for each cell by dividing the FRET intensity by the CFP intensity. The ratios for cells in a given strain were averaged to yield the Mean Ratio. The “FRET Value” of a donor-acceptor pair is the Mean Ratio normalized to that of the donor-only strain, as follows: FRET Value=[Mean Ratio (donor-acceptor)−Mean Ratio (donor only)]/ Mean Ratio (donor only). The FRET Value for the donor-only strain was therefore set to zero. The t-test was used to statistically compare the individual ratios from two given strains.

Fluorescence energy transfer between GFP derivatives in living yeast cells

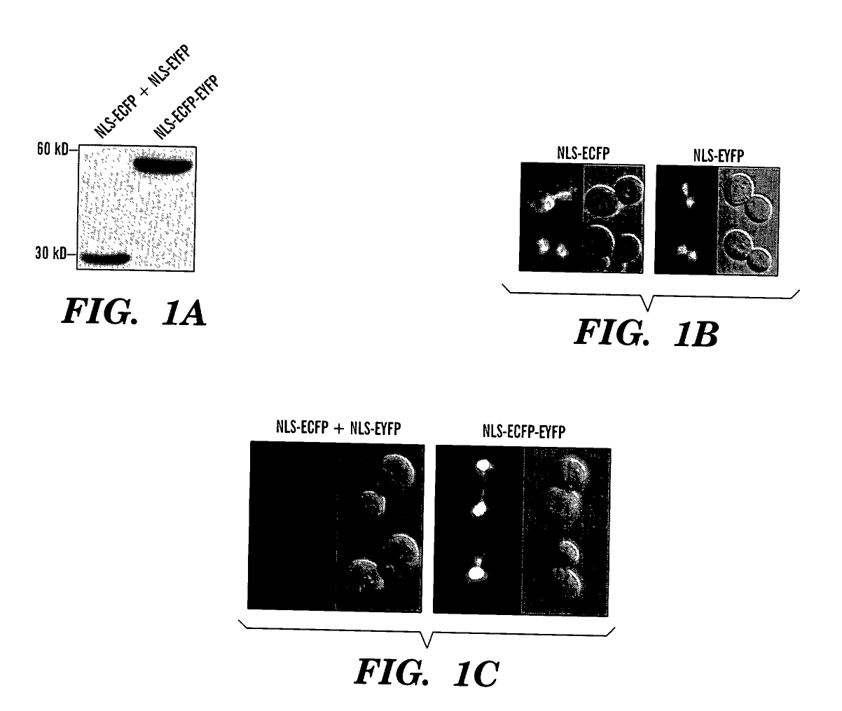

The feasibility of using FRET between ECFP and EYFP to assess protein interactions in yeast cells was established as follows. Plasmids were constructed encoding nuclear-targeted ECFP, EYFP, and a chimeric ECFP-EYFP, and introduced into S. cerevisiae. The production of intact proteins of the correct size was confirmed by immunoblot analysis with anti-GFP (FIG. 1A). NLS-ECFP and NLS-EYFP (29 kDa) and NLS-ECFP-EYFP (57 kDa) were expressed at approximately equal levels. When viewed by fluorescence microscopy, cells expressing NLS-ECFP or NLS-EYFP displayed a strong nuclear signal with the appropriate filter set (FIG. 1B), indicating that these GFP derivatives had the expected spectral characteristics in yeast. Energy transfer was assayed with a FRET filter set containing an ECFP excitation filter and an EYFP emission filter. In cells expressing both NLS-ECFP and NLS-EYFP, as verified with the CFP and YFP filter sets, no signal was observed with the FRET filter set (FIG. 1C, left panels). In contrast, cells expressing the chimeric NLS-ECFP-EYFP showed strong nuclear fluorescence when viewed with the FRET filters (FIG. 1C, right panels), demonstrating in vivo energy transfer from ECFP to EYFP. These data show that it is possible to detect GFP-based FRET in living yeast cells and that subcellular colocalization of ECFP and EYFP is not sufficient for energy transfer.

FRET reveals interactions between the importin Pse1 and several nucleoporins

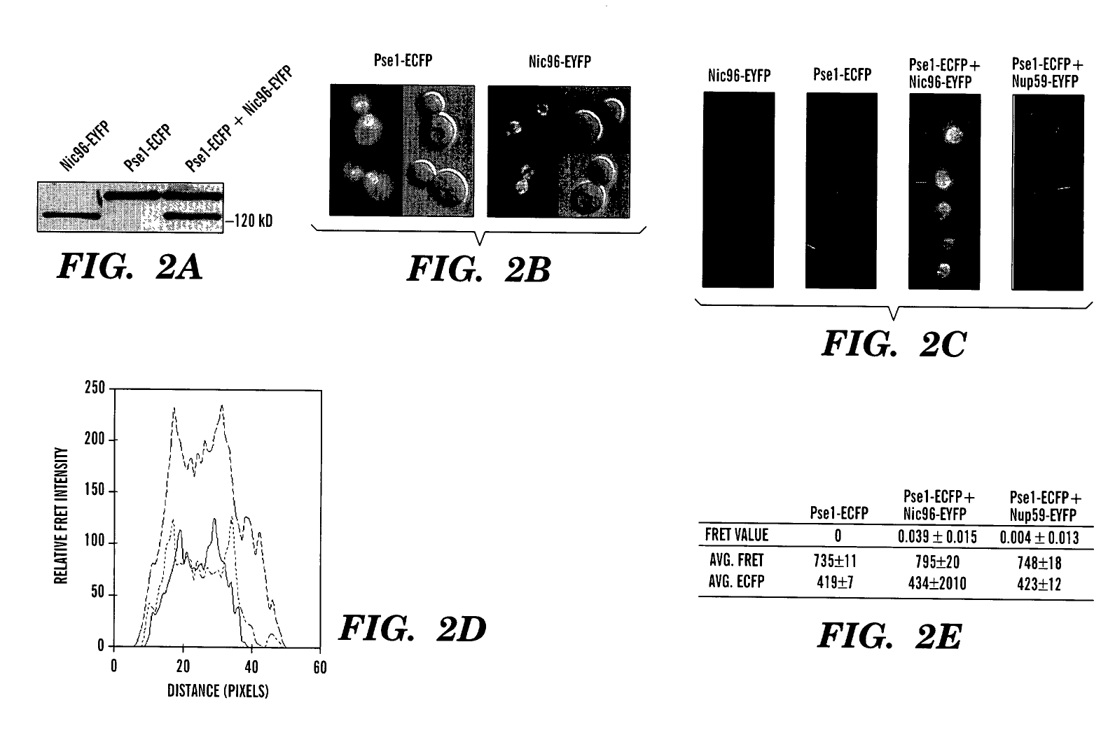

We used the ECFP-EYFP FRET system to investigate the interactions between transport receptors and nucleoporins in living yeast cells. To visualize one of the receptors, we fused ECFP to Pse1/Kap 121. Pse1 has previously been shown to import the transcription factor Pho4 (Kaffman et al., 1998) and to interact in a Ran-dependent manner with certain nucleoporins (Marelli et al., 1998; Seedorf et al., 1999). DNA encoding ECFP was integrated into the genome at the PSE1 locus, creating an open reading frame that encodes a full-length Pse1-ECFP fusion. Consequently, PSE1-ECFP is the only functional copy of PSE1 in the cells. Pse1-ECFP is expressed at the predicted size of 150 kDa, as confirmed by immunoblot analysis with anti-GFP (FIG. 2A). When viewed by fluorescence microscopy, Pse1-ECFP localizes predominantly at the nuclear envelope with some additional cytoplasmic and nuclear signal (FIG. 2B, left panels).

To visualize nucleoporins, DNA encoding EYFP was integrated into the genome at a particular NUP locus so that the cells expressed the Nup-EYFP fusion. As is the case for PSE1, the genomic integration creates a strain where NUP-EYFP replaces the endogenous gene. For example, NIC96, which is an essential nucleoporin gene, can be replaced by NIC96-EYFP, as evidenced by the expression of Nic96-EYFP at the predicted size of 125 kDa (FIG. 2A). When these cells are viewed by fluorescence microscopy, a punctate nuclear envelope signal is observed, indicating that Nic96-EYFP is properly localized at the nuclear envelope (FIG. 2B, right panels).

When Pse1-ECFP and Nic96-EYFP are expressed in the same cell, a FRET signal is obtained. A yeast strain co-expressing these fusion proteins was generated by integrating DNA encoding ECFP at PSE1 in the NIC96-EYFP strain. The resulting cells produce both fusions (FIG. 2A) and grow at the wildtype rate (data not shown). When these cells are viewed with the FRET filter set, a strong signal at the nuclear envelope is observed (FIG. 2C, Pse1-ECFP+Nic96-EYFP) suggesting an interaction between Pse1 and Nic96 at the NPC. Although cells expressing Pse1-ECFP alone show some leak-through ECFP emission when viewed with the FRET filters (FIG. 2C, Pse1-ECFP), this signal is substantially lower than that of cells co-expressing Pse1-ECFP and Nic96-EYFP. Cells expressing Nic96-EYFP show virtually no background signal (FIG. 2C, Nic96-EYFP).

The FRET assay was used to determine in vivo interactions between Pse1 and an extensive set of nucleoporins. Toward this end, EYFP was integrated to produce functional fusions to the nucleoporins Nup116, Nup120, Nup1, Nup2, Nup133, Nup145, Nup188, Nup82, Nup53, Nup59, Nup84, and Nup85. In each case, a fusion protein of the predicted size was expressed and located at the nuclear envelope (data not shown). The functionality of each Nup-EYFP fusion was assayed in a condition where that nucleoporin is essential for cell viability: Nup82 and Nup1 at 25° C.; Nup116, Nup145, Nup120, Nup84, Nup85, and Nup133 at 37° C,; Nup188-EYFP, Nup53-EYFP, and Nup59-EYFP in nup170; and Nup2-EYFP innup1-8.

FRET signal was observed between Pse1 and the nucleoporins Nup 116, Nup1, Nup2, Nup133, Nup145, Nup188, and Nup53, in addition to Nic96 (Table 1). These FRET results support previous evidence for physical interactions between Pse1 and Nup 116 (Seedorf et al., 1999) and between Pse1 and Nup53 (Marelli et al., 1998). No FRET signal was detected between Pse1 and Nup120, Nup82, Nup59, Nup84, or Nup85 (Table 1).

Digitized images captured with a charge-coupled device (CCD) camera were analyzed quantitatively to further determine that the FRET signal was significant. Intensity profiles along lines drawn through cells in FIG. 2C demonstrate that the FRET intensity is higher in Pse1-ECFP/Nic96-EYFP cells than in Pse1-ECFP/Nup59-EYFP and Pse1-ECFP cells, and show that in each case the profile peaks at two points corresponding to the nuclear envelope (FIG. 2D). Additionally, a quantitation assay employed two filter sets so that the FRET signal at the NPC could be normalized by the ECFP signal. The two filter-set system was sufficient to measure FRET in this case because the acceptor EYFP did not leak through the FRET filter set (FIG. 2C, Nic96-EYFP); the average FRET intensity at the nuclear envelope in Nic96-EYFP cells was only 0.6% higher than that in wildtype cells. The quantitation assay, described in Experimental Procedures, was initially performed on cells expressing Pse1-ECFP alone, Pse1-ECFP/Nic96-EYFP and Pse1-ECFP/Nup59-EYFP. The FRET Value for Pse1-ECFP/Nic96-EYFP represents a nearly 10-fold increase over that for Pse1-ECFP/Nup59-EYFP (FIG. 2E). In a t-test comparing the data for these strains, the p-value was<0.001, indicating that there is a specific and significant FRET interaction between Pse1-ECFP and Nic96-EYFP at the nuclear envelope. When the cytoplasmic regions in the same images were analyzed, the FRET Value in Pse1-ECFP/Nic96-EYFP cells was 0.013, below the threshold of a significant interaction.

This analysis was extended to the entire panel of strains expressing Pse1-ECFP and a nucleoporin-EYFP (Table 1). For strains in which FRET was observed, the FRET Values ranged from 0.018 to 0.039, representing significant interactions as indicated by p<0.05 in the t-test. The values for strains in which no FRET was detected ranged from 0.001 to 0.007.

Nucleoporin-protein fusions do not block the NPC

Photobleaching experiments demonstrated that tagging a nucleoporin with EYFP does not impede the passage of Pse1 through the NPC. If this were the case, an increased concentration of Pse1-ECFP at the nuclear envelope could, in principle, result in higher background signal that might be misconstrued as a FRET signal.

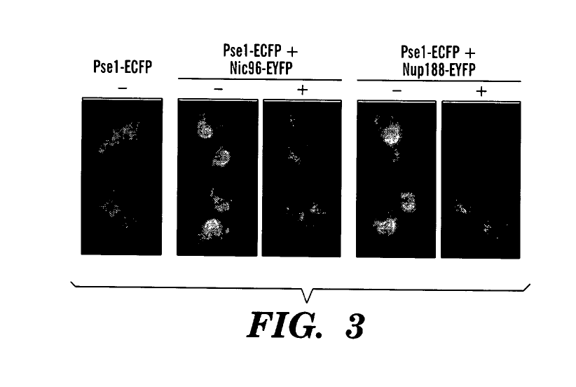

FRET signals in Pse1-ECFP/nucleoporin-EYFP cells were compared before and after the EYFP was deactivated by photobleaching. We hypothesized that a signal due to a genuine ECFP-EYFP interaction would disappear after EYFP photobleaching, whereas an artificial FRET signal due to an increased concentration of Pse1-ECFP at the nuclear envelope would be unaffected by EYFP photobleaching. Data is shown for Pse1-ECFP/Nic96-EYFP and Pse1-ECFP/Nup188-EYFP (FIG. 3). Images reflect the FRET signal either without treatment, or after a 15-second exposure to the YFP filter set that rendered the nucleoporin-EYFP signal undetectable. The signal in photobleached cells is reduced to the background level of Pse1 -ECFP. Similar results were obtained for all other Pse1-ECFP/nucleoporin-EYFP interactions (data not shown). In cells expressing only Pse1-ECFP, the fluorescence of Pse1-ECFP did not diminish after a 15-second exposure to the YFP filter set (data not shown), indicating that EYFP photobleaching has no effect on ECFP fluorescence. These results indicate that the FRET signals consist of EYFP sensitized emission and not ECFP leak-through emission, and that the signals are not an artifact of steric hindrance.

Pse1-NPC interaction depends on a functional nucleoporin

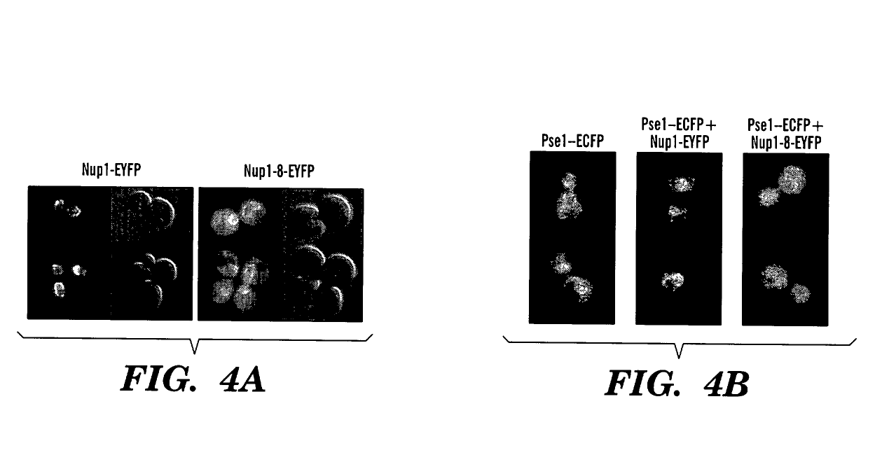

The interaction by FRET between Pse1-ECFP and Nupl-EYFP is not observed in strains harboring a mutation in NUP1. Genomically integrated EYFP fusions to NUP1 and to the N-terminal truncation mutant nup1-8 were created. When viewed with the YFP filter set, Nup1-EYFP localizes at the nuclear envelope (FIG. 4A, left panels). The mutant Nupl-8-EYFP, however, is observed both in the cytoplasm and at the nuclear envelope (FIG. 4A, right panels). This partial mislocalization is consistent with the results from fractionation experiments in which Nup1-8 was recovered in the soluble and insoluble fractions (Bogerd et al., 1994). To test the interaction of Nup1-8 with Pse1 in vivo, Pse1-ECFP was integrated into the nup1-8-EYFP and NUP1-EYFP strains. Western blotting showed that all protein fusions were of the expected sizes and expressed at equivalent levels (data not shown). Fluorescence microscopy demonstrated that the FRET signal at the nuclear envelope in Pse1-ECFP/Nup1-EYFP cells is higher than that in Pse1-ECFP cells, and that the signal is decreased in the Pse1-ECFP/Nup1-8-EYFP cells (FIG. 4B). This result indicates that the FRET signal depends directly on a functional Nupl-EYFP that is properly located at the NPC.

Physical interaction between Pse1and the nucleoporin Nic96

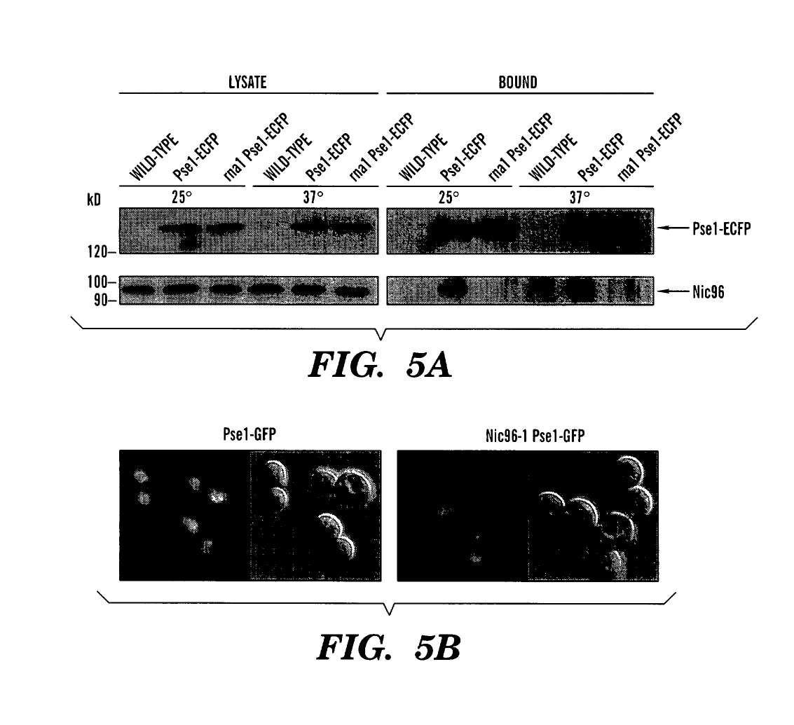

To assay the predictive value of the FRET experiments, we sought independent evidence for the novel interaction observed by FRET between Pse1 and Nic96. We immunoprecipitated Pse1-ECFP complexes from wild-type and temperature-sensitive rna1-1 cells. RNA1 encodes the activating protein for the Ran GTPase, and thus Ran-GTP levels are presumably high in the rna1-1 mutant (Corbett et al., 1995). Previously identified Pse1/nucleoporin interactions were not detectable in high levels of Ran-GTP (Marelli et al., 1998; Seedorf et al., 1999); Ran-dependence thus serves as a test for specificity. Wild-type cells, cells expressing Pse1-ECFP, and rna1-1 cells expressing Pse1-ECFP were grown at 25° C. to log phase, and then half of each culture was shifted to 37° C. Lysates were incubated with anti-GFP beads to isolate complexes containing Pse1-ECFP, and the bound fractions were probed for Nic96. Nic96 was found in complex with Pse1-ECFP, and this interaction was not detected in the rna1-1 mutant at 25° C. or 37° C. (FIG. 5A). This result further demonstrates the physical interaction between Pse1 and Nic96.

The relationship between Pse1 and Nic96 was investigated by examining the localization of Pse1-GFP in the mutant strain nic96-1 (Grandi et al., 1995). The Pse1-GFP signal at the nuclear envelope is substantially lower in nic96-1 compared to wild-type (FIG. 5B). Western blot analysis showed that Pse1-GFP is expressed at comparable levels in both strains (data not shown). These data indicate that Nic96 may be an important binding site for Pse1 during the translocation process.

Contacts Between an Exportin and Nucleoporins in vivo

The studies with the importin Pse1 were extended to the exportin Msn5. As with Pse1, the functionality of Msn5-ECFP was established, and the fusion was integrated into the nucleoporin-EYFP strains. The cells were then examined with the FRET filter set as described above and compared to cells expressing only Msn5-ECFP.

FRET signal was observed between Msn5 and Nic96, Nup 116, Nup1, Nup2, Nup133, Nup145, Nup188, Nup82, and Nup84, but not between Msn5 and Nup120, Nup53, Nup59, or Nup85 (Table 1). The FRET Values for strains in which FRET signal was observed ranged from 0.019 to 0.092, corresponding to p<0.05, whereas the values for strains in which no FRET was detected ranged from 0.001 to 0.007. Photobleaching experiments, as described above, confirmed that the FRET signals were due to sensitized emission. The patterns of Msn5 and Pse1 contacts thus are generally similar yet differ in the cases of Nup53, Nup82, and Nup84 (Table 1).

We detected interactions between the importin Pse1 and eight of the thirteen nucleoporins tested (Table 1). Importantly, two of these interactions have been previously observed by co-immunoprecipitation, supporting the FRET results. Moreover, the novel interaction between Pse1 and Nic96 was first identified by FRET and then independently confirmed by co-immunoprecipitation. These results confirm the predictive value of the FRET assay.

Nine nucleoporins from this study have previously been localized to specific regions of the NPC by immuno-electron microscopy (reviewed in Stoffler et al., 1999). Receptor-nucleoporin interactions identified by FRET, together with the localization of the nucleoporins, could have several implications for the translocation mechanism. For example, Nup145 has been isolated in a subcomplex that includes Nup120, Nup84, and Nup85 (Siniossoglou et al., 1996) and has been localized to the cytoplasmic filaments (Stoffler et al., 1999). Pse1 interacts by FRET with Nup145 but not Nup120, Nup84, or Nup85 (Table 1). Thus Nup145 could be a docking site for Pse1 on the cytoplasmic filaments at the start of an import event. However, the absence of a FRET signal has limited significance given the strict requirements for FRET, including the short distance between ECFP and EYFP and their proper relative orientation. Additionally, some interactions may be too transient even for detection by FRET.

The interaction between Pse1 and Nic96 is dependent on the nucleotide bound state of Ran. With Ran-GTP levels presumably high in the rna1-1 background, the Pse1-Nic96 interaction is no longer detected by co-immunoprecipitation (FIG. 5A). Nic96 has been localized to the central channel and to the end of the nuclear basket (Fahrenkrog et al., 1998). The localization of Nic96, the Ran-dependence of the Pse1 -Nic96 interaction, and the decreased steady-state levels of Pse1-GFP at the NPC in nic96-1 cells (FIG. 5B) offer the possibility that Nic96 is a terminal step in the Pse1 import pathway from which Pse1 is released by Ran-GTP for recycling to the cytoplasm.

Overlapping Translocation Pathways for an Importin and Exportin

We have identified multiple and distinct interactions between transport receptors and nucleoporins. In light of many parallel results from genetics, co-immunoprecipitations, and in vitro binding experiments, these interactions appear to reflect in vivo receptor-nucleoporin contacts. As such, these results have several implications for understanding the mechanism of translocation through the NPC.

The extensive set of receptor-nucleoporin interactions implies the existence of pathways through the NPC. The importin Pse1 and the exportin Msn5 share FRET contacts with seven of the nucleoporins in our panel (Table 1), suggesting that their pathways have substantial overlap. Interestingly, Pse1 and Msn5 share a substrate as well as several NPC contacts: the transcription factor Pho4 is imported by Pse1 and exported by Msn5. The overlap of different translocation pathways has been proposed previously (Kutay et al., 1997).

We also observed specificity in the patterns of interactions for Pse1 and Msn5. For cases in which an interaction is observed between a nucleoporin and one receptor but not another, the specificity permits a broader interpretation of the negative result; for example, no FRET was detected for Pse1/Nup82, but the FRET signal observed for Msn5/Nup82 indicates that the EYFP fused to Nup82 is generally accessible for energy transfer. The distinct Msn5/Nup82 contact is of special interest because Nup82 has been localized exclusively to the cytoplasmic face of the NPC (Fahrenkrog et al., 1998). Thus Nup82 may be a terminal release step in the Msn5 export pathway that interacts very transiently, or not at all, with the importin Pse1.

In general, the specific receptor-nucleoporin contacts in translocation pathways may serve as a regulatory mechanism for nuclear transport. For example, Marelli et al. (1998) have shown that Nup53 is phosphorylated during mitosis. We found that NupS3 interacts by FRET with Pse1 but not Msn5 (Table 1), suggesting that the change in Nup53 would specifically target the Pse1 pathway.

The translocation pathways described here include many non-essential nucleoporins. These results raise the question of how essential transport receptors such as Pse1 can rely on non-essential nucleoporins during translocation. One-dimensional diffusion of receptors along fibers composed of nucleoporins would allow for a degree of flexibility with regard to the fibers' composition (Koepp and Silver, 1996).

Our use of FRET to map protein-protein interactions in living cells to define a dynamic process is of general applicability, especially given the advantages of an expression system such as the yeast system. Screening large arrays of potential protein-protein interactions in vivo can readily be accomplished with FRET.

TABLE 1
FRET Values for interactions between transport
receptors and nucleoporins

| | Transport Receptor | |
|---|---|---|
| Nucleoporin | Pse1 | Msn5 |

| | | | | |
|---|---|---|---|---|
| Nic96 | +^{a} | (0.039) | + | (0.048) |
| Nup116 | + | (0.018) | + | (0.057) |
| Nup120 | nf^{b} | (0.001) | nf | (0.009) |
| Nup1 | + | (0.023) | + | (0.025) |
| Nup2 | + | (0.039) | + | (0.083) |
| Nup133 | + | (0.021) | + | (0.033) |
| Nup145 | + | (0.019) | + | (0.019) |
| Nup188 | + | (0.018) | + | (0.092) |
| Nup82 | nf | (0.003) | + | (0.021) |
| Nup53 | + | (0.025) | nf | (0.007) |
| Nup59 | nf | (0.004) | nf | (0.007) |
| Nup84 | nf | (0.007) | + | (0.021) |
| Nup85 | nf | (0.001) | nf | (0.001) |

^{a} Significant interaction as indicated by p < 0.05.
^{b} No FRET detected.

All references described herein are incorporated by reference.

1. A method to screen for and identify proteins that interact with a protein of interest comprising
(a) transforming an expression system, wherein the expression system comprises a plurality of cells that express a protein of interest tagged with one member of a fluorescent protein pair by a plurality of DNA molecules encoding a protein to be screened tagged with the other member of the fluorescent protein pair, wherein individual members of the plurality of cells are transformed by different DNA molecules encoding said protein to be screened;
(b) screening said transformed expression system by fluorescence resonance energy transfer (FRET) to determine whether an interaction has occurred; and
(c) identifying any cells within said transformed expression system where there has been a transfer of fluorescence between the fluorescent protein pair as indicating that an interaction between the protein of interest and a protein being screened has occurred.

2. The method of claim 1, wherein the plurality of cells are mammalian cells or yeast cells.

3. The method of claim 2 wherein the plurality of cells are yeast cells.

4. The method of claim 1, wherein the cells identified as having an interaction between the protein of interest and the protein being screened are isolated from the transformed expression system.

5. The method of claim 4, wherein the protein being screened is isolated from the cell.

6. The method of claim 1, 2, 3, 4 or 5, wherein the fluorescent protein pair is enhanced blue and green fluorescent proteins or enhanced cyan and yellow fluorescent proteins.

7. The method of claim 1, wherein a second group of cells is isogeneic to the plurality of cells of the expression system except that said cells have a bacterial, microbial or viral pathogen and said second group is screened to identify proteins that interact with the protein of interest, wherein one further identifies whether different proteins interact with the proteins of interest in the pathogen containing cells as compared to the cells of the expression system.

8. The method of claim 1, wherein a plurality of malignant cells also expresses the protein of interest and said cells are screened by the same method as used with the expression system to select and identify proteins that interact with the proteins of interest, in both groups and said proteins that interact with the protein of interest in said malignant cells are compared with the proteins that interact with the protein of interest in the plurality of cells of the expression system to identify whether different proteins interact in the malignant cell as compared to the plurality of cells of the expression system.
